(19) United States
(12) Patent Application Publication
(10) Pub. No.: US 2003/0008852 A1
(43) Pub. Date: Jan. 9, 2003

(54) Transdermal system containing acetylsalicylic acid for treatment of migraine

(76) Inventors: Franke Hanshermann, Wilstedt-Siedlung (DE); Bodo Asmussen, Bendorf-Sayn (DE)

Correspondence Address: BIRCH STEWART KOLASCH & BIRCH, PO BOX 747, FALLS CHURCH, VA, 22040-0747, US

(21) Appl. No.: 10/149,205

(22) PCT Filed: Dec. 1, 2000
(86) PCT No.: PCT/EP00/12092

(30) Foreign Application Priority Data
Dec. 11, 1999 (DE) 199 59 913.0

(51) Int. Cl. A61K 31/60

(52) U.S. Cl. 514/165

(57) ABSTRACT

The use of acetylsalicylic acid for the treatment of migraine and other serotonin-dependent, platelet-mediated disorders is characterized in that the active ingredient is administered with the aid of a transdermal therapeutic system (TTS).

The present invention relates to the use of acetylsalicylic acid for the treatment of migraine and other serotonin-dependent, platelet-mediated disorders, the active ingredient being administered via the transdermal route. The invention particularly encompasses methods in which acetylsalicylic acid is administered to the human skin by a transdermal therapeutic system for the purpose of prophylactic treatment of migraine. The invention further relates to the use of acetylsalicylic acid for the production of transdermal therapeutic systems for migraine prophylaxis, and to acetylsalicylic acid-containing transdermal therapeutic systems suitable for migraine prophylaxis.

Migraine is a multifactorial event triggered by various exogenous and endogenous causes. The underlying biochemical processes are largely known, although the pathophysiological processes have not been completely elucidated. A central role in migraine is ascribed to dysregulation of the cerebral blood flow. The latter is controlled by a number of different factors, e.g. biogenic amines, neuropeptides, prostaglandins inter alia. During a migraine episode there is initially strong vaso constriction, which may be mediated for example by serotonin. On the other hand, a marked increase in blood flow is recorded toward the end of the migraine episode. The initial increase in the serotonin level is attributed inter alia to an increased release of this biogenic amine by platelets.

The dysregulation of the cerebral/cranial blood flow leads—without a direct causal connection—eventually to the typical sensation of pain, which is referred to by the patients as “migraine headache”. This usually occurs episodically and in a pulsating manner and may considerably affect the general well-being of a person affected over a period of from a few hours up to some days. In addition, migraine attacks may be associated with diffuse autonomic complications and even neurological deficits.

The groups of active ingredients used for the pharmacotherapy of migraine are the following, in particular: non-steroidal antiinflammatory drugs (NSAID) and serotonin antagonists. The active ingredients are less suitable for prophylactic use because they need to be administered in high doses and/or have serious side effects. By contrast, they are used successfully for the treatment of severe and very severe forms of migraine. Prophylactic treatment of migraine is possible to date only by use of serotonin antagonists of the 5-HT2 type.

It is known from the literature that oral administration of acetylsalicylic acid (aspirin) in low doses (235 mg every second day) may exert a prophylactic effect on migraine attacks (J. E. Buring et al., JAMA Oct. 3, 1990, 264:13, 1711-1713). However, the reduction in the migraine recurrence rate observed with this low-dose therapy was small (20% compared with the placebo group). The use of aspirin for migraine prophylaxis is based on the assumption that, as a prostaglandin synthesis inhibitor, aspirin has an effect on the metabolism of particular celullar constituents of the blood, specifically the platelets, and reduces their biochemical reactivity. This may lead to a quantitative change in the neurotransmitters and hormones produced by these blood cells. Increased release of serotonin by platelets, as occurs at the start of a migraine episode, might be beneficially influenced by active ingredients such as aspirin.

Oral administration of aspirin entails various disadvantages. On the one hand, the biological half-life is rather short, because aspirin is rapidly hydrolylzed in the gastrointestinal tract to salicylic acid (SA) (G. Levy, “Clinical Pharmacokinetics of Aspirin”, Pediatrics 62, 867-872, 1978). However, the inhibition of platelet function—on which the migraine therapy also depends—is mediated by aspirin and not by SA (W. Horsch, “Die Salicylate”, Pharmazie 34, 585-604, 1979). This means that a considerable part of the administered dose is not utilized. On the other hand, oral administration of aspirin, especially when this extends over prolonged periods, frequently leads to gastrointestial side effects, e.g. gastric hemorrhages.

Aspirin-containing transdermal therapeutic systems (TTS) which make it possible to administer aspirin avoiding the gastrointestinal tract have already been described, e.g. in DE 43 32 093 C2 and in DE 42 41 128 C2. The latter publication refers in particular to the use for antithrombotic therapy and for prophylaxis of colon cancer. In addition, topical or transdermal use of aspirin, also in the form of ointments, gels and the like, has also been described for the therapeutic treatment of various other pathological states, e.g. for rheumatism (Chen et al.; Zhongguo Yiyuan Yaoxue Zazhi vol. 11, 245-247, 1991), for analgesic or antipyretic indications, or for suppression of inflammation (U.S. Pat. No. 3,598,122; FR-M 1757; FR-A 2 297 612; U.S. Pat. No. 4,219,548; EP-A 0055635; JP-A 1,242,521).

It was an object of the present invention to indicate a method for the pharmacological prophylaxis of migraine which has few side effects and is therefore suitable for long-term uses, which is simple and convenient for the patient to use and, at the same time, is effective in preventing migrainous states but which, on the other hand, does not have the known disadvantages associated with oral administration of aspirin.

The object is achieved according to the invention by administration of acetylsalicylic acid by the transdermal route, preferably using a transdermal therapeutic system of the invention. It has surprisingly emerged from series of clinical tests with the transdermal administration system of the invention that the frequency of migraine is significantly reduced. It is indicative in this connection that—unlike oral administration—the aspirin level in the blood plasma remains low and does not exceed 0.5 μg/ml (cf. example 3). It is possible to achieve average acetylsalicylic acid plasma levels below 10 ng/ml over the course of a day. Despite these low plasma levels of aspirin, the threshold for triggering migraine episodes was surprisingly found to be raised. The avoidance of high plasma levels of aspirin also reduces the risk of systemic side effects.

It is possible with the aspirin-containing TTS employed according to the invention for migraine prophylaxis to achieve average blood plasma salicylate levels of at least 20 ng/ml, preferably from 100 to 400 ng/ml, in humans over the course of a day during the treatment (cf. example 3). These values indirectly suggest a very efficient uptake of aspirin through the skin.

Thus the present invention makes an effective and low-cost treatment method which has few side effects and is convenient for the patient or user possible for the prophylaxis of migraine.

The therapeutic method proposed by the invention is suitable both for long-term prophylaxis and for acute prophylaxis of migraine. It is moreover possible to combine the transdermal administration of the invention from TTS with conventional migraine prophylaxis and other therapy regimens. Although the present invention is primarily directed at the treatment or prophylaxis of migraine, this does not exclude the possibility of also using it for the treatment of other serotonin-dependent, platelet-mediated disorders.

It may prove advantageous in certain use situations for acetylsalicylic acid to be administered in accordance with a particular embodiment of the invention in combination with one or more other active ingredients and/or in combination with excipients. Other active ingredients which are particularly suitable are those which likewise have a pain-relieving effect and are amenable to transdermal uptake. Active ingredients preferably considered for a combined administration with aspirin in the TTS are from the following groups: serotonin antagonists, nonselective serotonin derivatives, single analgesics, analgesic combinations, ergotamine derivatives, non-steroidal antiinflammatory drugs (NSAID), corticosteroids, phenothiazines, opiate analgesics, beta-blockers, calcium channel blockers, tricyclic antidepressants, antiepileptics and monoamine oxidase inhibitors.

The aspirin-containing TTS are preferably used in such a way that the duration of application, relating to a single TTS, is not more than one week, preferably 1 to 3 days. Continuous daily use over a period of at least 16 hours is particularly advantageous in this connection. The amount of aspirin delivered to the skin by the TTS within one day is preferably in a range between 1 mg and 100 mg.

Particularly suitable TTS which can be employed according to the invention for migraine prophylaxis using aspirin as active ingredient are TTS of the matrix type, which have a structure composed of a backing layer which is essentially impermeable to active ingredient and moisture, of one or more active ingredient-containing matrix layer(s), and of a detachable protective layer. It is moreover possible advantageously to use embodiments in which acetylsalicylic acid are present predominantly in crystalline form in at least one of the matrix layers, and in which at least part of the active ingredient acetylsalicylic acid is in crystalline form as a stable anhydrous modification which melts above 132° C. Aspirin crystals with a diameter below about 50-100 μm are particularly advantageous.

The TTS of the invention may, beside acetylsalicylic acid, comprise other active ingredients which have already been mentioned above. These may be present either together with acetylsalicylic acid in the same matrix layer, or in one or more separate matrix layers.

Embodiments which are particularly preferred are those in which the active ingredients are present in at least two matrices separate from one another and are delivered with release rates which are independent of one another.

The skin permeation rates, based on aspirin, obtainable with the aspirin-containing TTS of the invention are preferably in the range 0.02-2 mg/cm²d, particularly preferably in the range 0.1-0.4 mg/cm²d.

The basic material employed for a matrix of the TTS of the invention comprises in particular acrylic ester-containing copolymers, and additionally mixtures of rubbers and resins, polyvinyl acetate, silicone polymers and many other materials suitable for use on the human skin.

The active ingredient-containing polymer matrix may additionally comprise excipients and additives, e.g. fillers such as titanium dioxide, zinc oxide, chalk, activated carbon, finely divided silica, and skin-permeation promoting additives known to the skilled worker. These include, for example, liquid additives such as short-chain alcohols, triglycerides, cholesterol, cineol, delta-tocopherol, diethylene glycol, diethylene glycol monoethyl ether, diisopropyl adipate, dimethyldecylphosphoxide, dimethylisosorbide, dimethyllauroylamide, dimethyl sulfoxide, dodecyl sulfoxide, acetic acid, ethyl acetate and other aromatic and aliphatic esters, ethylene glycol, ethylene glycol monolaurate and other esters and ethers of ethylene glycol and propylene glycol, 2-octyldodecanol, low-viscosity paraffin, glycerol, glycerol monooleate, glycerol monostearate, hydrogenated castor oil, isopropyl myristate, isopropyl palmitate, lauric acid diethanolamide, menthol or other volatile terpene derivatives (which are constituents of the mixtures of many natural essential oils), methyl benzoate, methyl octyl sulfoxide, mono- or diethylacetamide, N,N-diethyl-m-toluamide, 1-octanol and other volatile medium chain-length alcohols, octanoic acid and other medium chain-length aliphatic carboxylic acids, oleyl alcohol, olive oil, oleic acid, oleyl oleate, phenylethanol, propylene glycol, ricinoleic acid, triacetin, and mixtures of said substances. However, in this connection, the ability of the active ingredient acetylsalicylic acid to react with esters and acids, and alcohols, must be taken into account in a few cases, which limits the use of these substances.

Numerous synthetic materials distinguished by strength and diffusion resistance are suitable for forming the backing layer, especially polyesters, polyvinyl chloride, ethylene/vinyl acetate, vinyl acetate, polyethylene, polypropylene, cellulose derivatives and many others. In a few cases, vapor deposition of metals or other diffusion-blocking additives, such as silica, alumina or the like, on the backing layer can be carried out. It is also possible to improve acceptance by putting a skin-colored coating on the outside of the backing layer or treating it in another way in order to improve the appearance. The detachable protective layer which is to be removed before application of the TTS to the skin may be produced from polyester material but also from any other synthetics suitable for use on the skin, e.g. from polyvinyl chloride, ethylene/vinyl acetate, vinyl acetate, polyethylene, polypropylene, cellulose derivatives and many others. As in the production of the backing layer it is also possible for the protective layer to undergo additional vapor deposition of diffusion-blocking substances.

The invention is explained in more detail by means of examples below.

### EXAMPLE 1

Composition of the Matrix Layer of a TTS of the Invention

| | |
|---|---|
| Acetylsalicylic acid | 19.05% |
| Durotak ® 318-2052 | 60.32% |
| Lemon oil | 12.38% |
| Plastoid ® B | 8.25% |

The data signify the respective proportions by weight based on the total weight of the active ingredient matrix.

### EXAMPLE 2

In-vitro Release of Aspirin from a TTS of the Invention.

The aspirin release was determined using the USP paddle over disk method.

Amount of aspirin released [mg/50 cm² TTS]

| Time [h] | Mean (n = 6) | Standard deviation |
|---|---|---|
| 0 | 0 | 0 |
| 4 | 57.5 | 0.96 |
| 7 | 80.7 | 0.96 |
| 24 | 131.5 | 1.41 |

Plasma Levels after Transdermal Administration

In a clinical study, two systems of the invention (each loaded with 84 mg of aspirin) were stuck onto the skin of each of four male subjects, changing each day over a period of 14 days.

On days 10 and 14, the content of acetylsalicylic acid and salicylic acid in the blood plasma was determined by gc-ms: Whereas the content of acetylsalicylic acid was below the limit of determination of 6 ng/ml at both times of measurement, the salicylic acid level was 72 i 18 ng/ml on day 10 and 157 ng/ml on day 14.

1. The use of acetylsalicylic acid for the treatment of migraine and other serotonin-dependent, platelet-mediated disorders, characterized in that the active ingredient is administered with the aid of a transdermal therapeutic system (TTS).

2. The use of acetylsalicylic acid for producing a TTS for the treatment of migraine and other serotonin-dependent, platelet-mediated disorders.

3. A method for the treatment of migraine and other serotonin-dependent, platelet-mediated disorders, characterized in that it is based on the transdermal administration of acetylsalicylic acid by means of a TTS.

4. The use as claimed in claim 1 or 2, or method as claimed in claim 3, characterized in that an elevated acetyl-salicylic acid level has not built up in the blood plasma during the treatment, but in any event the plasma level of 0.5 μg/ml is not exceeded.

5. The use or method as claimed in one or more of the preceding claims, characterized in that average blood plasma salicylate levels of at least 20 ng/ml, preferably of from 100 to 400 ng/ml, are achieved in humans over the course of a day during the treatment.

6. The use or method as claimed in one or more of the preceding claims, characterized in that it takes place for the purpose of migraine prophylaxis, preferably for the purpose of long-term or acute prophylaxis of migraine.

7. The use or method as claimed in one or more of the preceding claims, characterized in that the transdermal administration takes place in addition to conservative migraine prophylaxis and other therapeutic regimens.

8. The use or method as claimed in one or more of the preceding claims, characterized in that acetylsalicylic acid is administered in combination with one or more other active ingredients and/or in combination with excipients.

9. The use or method as claimed in claim 8, characterized in that the other active ingredient(s) is or are selected from the group consisting of serotonin antagonists, nonselective serotonin derivatives, single analgesics, analgesic combinations, ergotamine derivatives, non-steroidal antiinflammatory drugs (NSAID), corticosteroids, phenothiazines, opiate analgesics, beta-blockers, calcium channel blockers, tricyclic antidepressants, antiepileptics and monoamine oxidase inhibitors.

10. The use or method as claimed in one or more of the preceding claims, characterized in that the duration of application, relating to a single TTS, is not more than one week, preferably 1 to 3 days, with particular preference for continuous daily use over a period of at least 16 hours.

11. The use or method as claimed in one or more of the preceding claims, characterized in that the TTS delivers at least 1 mg and at most 100 mg of acetylsalicylic acid per day to the skin.

12. The use or method as claimed in one or more of the preceding claims, characterized in that the acetylsalicylic acid-containing TTS makes skin permeation rates in the range 0.02-2 mg/cm²d, preferably in the range 0.1-0.4 mg/cm²d, possible.

13. A transdermal therapeutic system for administration of acetylsalicylic acid for migraine treatment as claimed in one or more of the aforementioned claims, characterized in that it has a structure composed of a backing layer which is essentially impermeable to active ingredient and moisture, of one or more active ingredient-containing matrix layer(s), and of a detachable protective layer, and in that at least one of the matrix layers comprises acetylsalicylic acid.

14. A transdermal therapeutic system as claimed in claim 13, characterized in that at least one matrix layer comprises the active ingredient acetylsalicylic acid predominantly in crystalline form, and in that at least part of the active ingredient acetylsalicylic acid is in crystalline form as a stable, anhydrous modification which melts above 132° C.

15. A transdermal therapeutic system as claimed in claim 13 or 14, characterized in that it comprises one or more other active ingredients and/or excipients, where the other active ingredient(s) is or are preferably selected from the group consisting of serotonin antagonists, nonselective serotonin derivatives, single analgesics, analgesic combinations, ergotamine derivatives, non-steroidal antiinflammatory drugs (NSAID), corticosteroids, phenothiazines, opiate analgesics, beta-blockers, calcium channel blockers, tricyclic antidepressants, antiepileptics and monoamine oxidase inhibitors.

16. A transdermal therapeutic system as claimed in any of claims 13-15, characterized in that the active ingredients are delivered with release rates which are independent of one another in at least two matrices separate from one another.

17. A transdermal therapeutic system as claimed in any of claims 13-16, characterized in that the rate of delivery of acetylsalicylic acid is at least 1 mg per day and at most 100 mg per day.

18. A transdermal therapeutic system as claimed in any of claims 13-17, characterized in that it makes skin permeation rates in the range 0.02-2 mg/cm²d, preferably in the range 0.1-0.4 mg/cm²d, possible.
